SEQUENCE LISTING (14 sequences)

SEQ ID NO: 1 - protein, 393 aa, Homo sapiens [sequence omitted]

SEQ ID NO: 2 - DNA, 2603 bp, Homo sapiens
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 3 - DNA, 19 bp, Artificial sequence
  synthetic
gtgctccccactttggaac

SEQ ID NO: 4 - DNA, 22 bp, Artificial sequence
  synthetic
ccaggcttctaagtaccctgag

SEQ ID NO: 5 - DNA, 20 bp, Artificial sequence
  synthetic
tcatcccttcctccctcttt

SEQ ID NO: 6 - DNA, 20 bp, Artificial sequence
  synthetic
ctcttaaggccattgctcca

SEQ ID NO: 7 - DNA, 21 bp, Artificial sequence
  synthetic
ccttcctccctctttctttca

SEQ ID NO: 8 - DNA, 20 bp, Artificial sequence
  synthetic
aggctgagagggtgtcacat

SEQ ID NO: 9 - DNA, 14 bp, Homo sapiens
ctggagatcaaacc

SEQ ID NO: 10 - DNA, 9 bp, Homo sapiens
gagtgcaac

SEQ ID NO: 11 - DNA, 9 bp, Homo sapiens
gtgggcttc

SEQ ID NO: 12 - DNA, 20 bp, Artificial sequence
  synthetic
tgacttgtgctccccacttt

SEQ ID NO: 13 - DNA, 1181 bp, Homo sapiens
atgcccaagaagaagccgacgcccatccagctgaacccggcccccgacggctctgcagttaacgggaccagctctgcggagaccaacttggaggccttgcagaagaagctggagggctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 14 - DNA, 21 bp, Artificial sequence
  synthetic
gtccccaggcttctaagtacc

(19) United States
(12) Patent Application Publication
(10) Pub. No.: US 2018/0010103 A1
(43) Pub. Date: Jan. 11, 2018

(54) METHODS AND BIOMARKERS FOR DETECTION AND TREATMENT OF LANGERHANS CELL HISTIOCYTOSIS

(71) Applicant: The Regents of the University of Michigan, Ann Arbor, MI (US)

(72) Inventors: Kojo Elenitoba-Johnson, Ann Arbor, MI (US); Mark J. Kiel, Ann Arbor, MI (US); Helmut Weigelin, Ann Arbor, MI (US); Noah A. Brown, Ann Arbor, MI (US); Bryan L. Betz, Ann Arbor, MI (US); Megan Lim, Ann Arbor, MI (US)

(21) Appl. No.: 15/547,327

(22) PCT Filed: Jan. 29, 2016
(86) PCT No.: PCT/US2016/015712

§ 371 Date: Jul. 28, 2017

Related U.S. Application Data
(60) Provisional application No. 62/110,035, filed on Jan. 30, 2015.

(51) Int. Cl. C12N 9/12; G01N 33/574; C12Q 1/68

(52) U.S. Cl. CPC C12N 9/1205; C12Q 1/6883; C12Y 207/12002; G01N 33/574; C12Q 1/6858; C12Q 2600/118; C12Q 2600/158; C12Q 2600/156

(57) ABSTRACT

The present invention relates to methods and biomarkers for detection and characterization of Langerhans cell histiocytosis in biological samples (e.g., tissue samples, blood samples, plasma samples, cell samples, serum samples). In particular, the present invention provides compositions and methods for diagnosing a patient as having a Langerhans cell histiocytosis by identifying mutations in the MAP2K1 gene or gene products.

### CROSS-REFERENCE TO RELATED APPLICATIONS

The present application claims the priority benefit of U.S. Provisional Patent Application 62/110,035, filed Jan. 30, 2015, which is incorporated by reference in its entirety.

### FIELD OF THE INVENTION

The present invention relates to methods and biomarkers for detection and characterization of Langerhans cell histiocytosis in biological samples (e.g., tissue samples, blood samples, plasma samples, cell samples, serum samples). In particular, the present invention provides compositions and methods for diagnosing a patient as having a Langerhans cell histiocytosis by identifying mutations in the MAP2K1 gene or gene products.

### BACKGROUND OF THE INVENTION

Langerhans cell histiocytosis (LCH) is characterized by a clonal proliferation of specialized cells with characteristics resembling antigen presenting cells that reside in the skin and mucosa (see, e.g., Jaffe R, et al., Tumours derived from Langerhans cells. In: World Health Organization Classification of Tumours of the Haematopoietic and Lymphoid Tissues. Lyon: IARC; 2008:358-360). The disease manifests a broad clinical spectrum ranging from focal and self-limited disease to aggressive multisystem disease with 20% mortality (see, e.g., Minkov M. Paediatr Drugs. 201;13(2):75-86). BRAF V600E mutations have been identified in 38% to 57% of LCH cases (see, e.g., Badalian-Very G, et al. Blood. 2010; 116(11):1919-23; Sahm F, et al., Blood. 2012; 120(12):e28-34). This mutation results in constitutive activation of the mitogen-activated protein kinase (MAPK) pathway. Since this discovery, patients with BRAF V600E mutated LCH have been successfully treated with BRAF inhibitors (see, e.g., Haroche J, et al., Blood. 2013; 121(9):1495-1500). It has been noted that the intensity of Langerhans cell immunohistochemical staining for phosphorylated downstream mediators of the MAPK pathway did not vary with BRAF mutation status (see, e.g., Badalian-Very G, et al. Blood. 2010; 116(11):1919-23). This finding suggests another mechanism of MAPK pathway activation in BRAF wild-type cases.

Additional insight into the pathogenesis of Langerhans cell histiocytosis is needed. For example, identification of mutations that may contribute to the pathogenesis of LCH in cases without BRAF V600E mutations are needed.

### SUMMARY OF THE INVENTION

Langerhans cell histiocytosis (LCH) represents a clonal proliferation of Langerhans cells. BRAF V600E mutations have been identified in approximately 50% of cases. To discover other genetic mechanisms underlying LCH pathogenesis, experiments conducted during the course of developing embodiments for the present invention studied 8 cases of LCH using a targeted next generation sequencing platform. An E102_I103del mutation in MAP2K1 was identified in one BRAF wild-type case and confirmed by Sanger sequencing. Analysis of 32 additional cases using BRAF V600E allele-specific PCR and Sanger sequencing of MAP2K1 exons 2 and 3 revealed somatic, mutually exclusive BRAF and MAP2K1 mutations in a total of 18/40 (45.0%) and 11/40 (27.5%) cases, respectively. This is the first report of MAP2K1 mutations in LCH which occur in 50% of BRAF wild-type cases. The mutually exclusive nature of MAP2K1 and BRAF mutations implicates a critical role of oncogenic MAPK signaling in LCH. Such findings additionally have implications for the use of BRAF and MEK inhibitor therapy.

Accordingly, the present invention relates to methods and biomarkers for detection and characterization of Langerhans cell histiocytosis (LCH) in biological samples (e.g., tissue samples, blood samples, plasma samples, cell samples, serum samples). In particular, the present invention provides compositions and methods for diagnosing a patient as having a Langerhans cell histiocytosis by identifying mutations in the MAP2K1 gene or gene products.

In one aspect, the invention provides a method for diagnosing LCH in an individual by: a) evaluating a sample containing nucleic acids from the individual for the presence or absence of one or more mutations in MAP2K1 mutations (e.g., one or more of the nucleic acid MAP2K1 mutations shown in Table 2) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T); and b) identifying the individual as having a LCH when the MAP2K1 nucleic acid comprises at least one mutation (e.g., one or more of the nucleic acid MAP2K1 mutations shown in Table 2) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T).

The sample may be any suitable biological sample including, for example, whole blood (i.e., MAP2K1 nucleic acid being extracted from the cellular fraction), plasma, serum, and tissue samples (e.g., biopsy and paraffin-embedded tissue). The MAP2K1 nucleic acid may be any convenient nucleic acid type including, for example, genomic DNA, RNA (e.g., mRNA), or cDNA prepared from subject RNA. Alternatively, the MAP2K1 nucleic acid mutation may be inferred by assessing the MEK1 protein (encoded by MAP2K1 gene) from the individual. For example, identification of a mutant MEK1 protein is indicative of a mutation in the MAP2K1 gene. Suitable detection methodologies include oligonucleotide probe hybridization, primer extension reaction, nucleic acid sequencing, and protein sequencing. In some embodiments, the individual is screened for the presence of a pathological BRAF mutation either simultaneously or prior to screening for the MAP2K1 nucleic acid mutation. In some embodiments, individuals are first identified as lacking a pathological BRAF mutation (e.g., the V600E mutation) and subsequently screened for an MAP2K1 nucleic acid mutation.

The invention also provided oligonucleotides (e.g., primers and probes) suitable for assessing MAP2K1 nucleic acid mutations. For example, suitable probes are designed to specifically hybridize to a nucleotide sequence containing at least one MAP2K1 mutation disclosed herein (i.e., but not hybridize to a non-mutated sequence). Suitable primers include allele-specific primers and primers suitable for primer extension reactions (e.g., SNaPShot® primers). The invention also provides antibodies that specifically bind to mutated MEK1 proteins encoded by the mutated MAP2K12 nucleic acids disclosed herein.

The invention also provides a method of assessing the LCH status of an individual, comprising: (a) evaluating a sample containing nucleic acids from the individual for the presence or absence of one or more mutations in both alleles of the MAP2K1 gene (e.g., one or more of the nucleic acid MAP2K1 mutations shown in Table 2) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T); and (b) identifying the individual (i) as having LCH or being predisposed to LCH when the individual is homozygous for one of such mutations (ii) as being predisposed to LCH when the individual is heterozygous for one of such mutations, or (iii) as having no predisposition to LCH caused by one of such mutations when each of such mutations is absent from both alleles of the MAP2K1 gene.

In one embodiment, the nucleic acid from the individual is RNA. In a further embodiment, evaluating comprises amplifying MAP2K1 nucleic acid and performing sequencing analysis of the amplified nucleic acid.

In another aspect, the invention provides a method of assessing the LCH status of an individual, comprising: (a) evaluating a sample containing MEK1 protein from the individual for the presence or absence of one or more MEK1 protein mutations and wild type MEK1 protein, such protein mutations being encoded by mutations within the MAP2K1 gene (e.g., one or more of the amino acid and/or nucleic acid MAP2K1 mutations shown in Table 2) (e.g., one or more of the following amino acid sequence MEK1 mutations (in comparison to wild type MEK1 amino acid sequence (SEQ ID NO: 1)): R47Q; R49C; F53_Q58delinsL; K57_G61del; 199_K104del; H100_I103delinsPL; E102_I103del; A106T; C121S; and G128V) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T); and (b) identifying the individual (i) as having LCH or being predisposed to LCH when the sample shows the presence of one or more of such protein mutations and the absence wild type MEK1 protein, (ii) as being predisposed to LCH when the individual shows the presence of wild type MEK1 protein and one or more of such protein mutations, or (iii) as having no predisposition to LCH when the individual shows the absence of each of such protein mutations.

In one embodiment of the foregoing aspect, evaluating comprises using antibodies against wild type MEK1 protein and each of the protein mutations encoded by mutations within the MAP2K1 gene (e.g., one or more of the amino acid and/or nucleic acid mutations shown in Table 2) (e.g., one or more of the following amino acid sequence MEK1 mutations (in comparison to wild type MEK1 amino acid sequence (SEQ ID NO: 1)): R47Q; R49C; F53_Q58delinsL; K57_G61del; 199_K104del; H100_I103delinsPL; E102_I103del; A106T; C121S; and G128V) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T). In another embodiment, evaluating comprises using protein sequencing.

In yet another aspect, the invention provides a method of identifying an individual with an increased likelihood of having LCH, comprising: (a) evaluating a sample containing nucleic acids from the individual for the presence or absence of one or more mutations in both alleles of the MAP2K1 gene (e.g., one or more of the nucleic acid MAP2K1 mutations shown in Table 2) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T); and (b) identifying the individual as having an increased likelihood of having LCH when one of such mutations is present in at least one allele.

In one embodiment of the foregoing aspect, the nucleic acid from the individual is RNA. In a further embodiment, evaluating comprises sequencing the MAP2K1 nucleic acid.

In still another aspect, the invention provides a method of identifying an individual with an increased likelihood of having LCH, comprising: (a) evaluating a sample containing protein from the individual for the presence or absence of one or more MEK1 protein mutations encoded by mutations within the MAP2K1 gene (e.g., one or more of the amino acid and/or nucleic acid mutations shown in Table 2) (e.g., one or more of the following amino acid sequence MEK1 mutations (in comparison to wild type MEK1 amino acid sequence (SEQ ID NO: 1)): R47Q; R49C; F53_Q58delinsL; K57_G61del; 199_K104del; H100_I103delinsPL; E102_I103del; A106T; C121S; and G128V) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T); and (b) identifying the individual as having an increased likelihood of having LCH when one of said mutations is present.

In one embodiment of the foregoing aspect, evaluating comprises using antibodies against wild type MEK1 protein and each of the protein mutations encoded by the MAP2K1 gene. In another embodiment, evaluating comprises using protein sequencing.

In an embodiment of any of the foregoing aspects, the sample is selected from the group consisting of blood, serum, and plasma.

In another embodiment of any of the foregoing aspects, evaluating comprises amplifying MAP2K1 nucleic acid and hybridizing the amplified nucleic acid with a detection oligonucleotide that is capable of specifically detecting BRAF nucleic acid under hybridization conditions. Another embodiment further comprises said individual not having a pathologic mutation in the BRAF gene. Another embodiment further comprises said individual not having a mutation in the BRAF gene encoding V600E mutation.

In an embodiment of any of the foregoing aspects, “subject” and/or “patient” refers to a human (e.g., a human being screened for LCH) (e.g., a human at risk for developing LCH).

In an embodiment of any of the foregoing aspects, the methods and uses further comprise the step of treating the subject having one or more mutations in the MAP2K1 gene (e.g., one or more of the nucleic acid MAP2K1 mutations shown in Table 2) (e.g., one or more of the following nucleic acid sequence MAP2K1 mutations (in comparison to wild type MAP2K1 nucleic acid sequence (SEQ ID NO: 2)): 140G>A; 145C>T; 159_173del; 168_182del; 295_312del; 299_307delinsCTC; 303_308del; 316G>A; 304_309del; 361T>A; and 383G>T) for LCH.

In some embodiments, the treating comprises radiation therapy.

In some embodiments, the treating comprises chemotherapy (e.g., alkylating agents, antimetabolites, vinca alkaloids, etc.).

In some embodiments, the treating comprises inhibiting MEK1 expression and/or activity. In some embodiments, inhibiting MEK1 expression and/or activity is accomplished through administration of an agent configured to inhibit MEK1 expression and/or activity. Applicable examples of MEK1 inhibiting agents include, but are not limited to, Selumetinib (AZD6244) (see, e.g., Nature, 2012, 487(7408):505-9; Nature, 2010, 468(7326):973-7; Nature, 2010, 468(7326):968-72), PD0325901 (see, e.g., Nature, 2014, 10.1038/nature13887; Cell, 2012, 151(5):937-50; Nat Methods, 2011, 8(6):487-93), Trametinib (GSK1120212) (see, e.g., Nature, 2014, 510(7504):283-7; Nature, 2014, 10.1038/nature13887; Nature, 2014, 508(7494):118-22), U0126-EtOH (see, e.g., Cell, 2013, 153(4):840-54; Nat Genet, 2011, 44(2):133-9), PD184352 (C1-1040) (see, e.g., Science, 2011, 331(6019):912-6; Nat Genet, 2011, 44(2):133-9; Cancer Discov, 2013, 3(9):1058-71), Refametinib (RDEA119, Bay 86-9766), PD98059 (see, e.g., J Natl Cancer Inst, 2012, 104(21):1673-9; Hepatology, 2013, 59(4):1262-72; Sci Signal, 2011, 4(192):ra62), BIX 02189 (see, e.g., Anat Cell Biol, 2011, 44(4):265-73; Biochim Biophys Acta, 2014, 1843(5):945-54; Am J Pathol, 2013, 183(6):1758-68), Binimetinib (MEK162, ARRY-162, ARRY-438162) (see, e.g., Mol Oncol, 2014, 8(3):544-54), Pimasertib, SL-327, BIX 02188 (see, e.g., Mol Cancer, 2014, 13:40; Exp Neurol, 2012, 238(2):209-17), AZD8330 (see, e.g., Cell, 2012, 32(34):4034-42), TAK-733 (see, e.g., Mol Cancer Ther, 2014, 13(2)), Honokiol, and PD318088.

Additional embodiments will be apparent to persons skilled in the relevant art based on the teachings contained herein.

### DESCRIPTION OF THE DRAWINGS

FIG. 1A-D: Somatic MAP2K1 mutations in Langerhans cell histiocytosis. (A) The frequency of mutually exclusive BRAF and MAP2K1 mutations in Langerhans cell histiocytosis is shown. (B) A portion of the MAP2K1 gene including exons 2 and 3 is depicted below and regions of the MEK1 protein encoded by exons 2 and 3 are depicted above. Somatic mutations in Langerhans cell histiocytosis involve the N-terminal negative regulatory region encoded by exon 2 and the catalytic core encoded by exon 3. Circles above the protein designate substitutions and bars below the protein indicate in-frame deletions. (C) A sequence electropherogram from case 26 (top) demonstrates the most common mutation identified in this study, E102_I103del. The absence of this mutation in a sequence electropherogram from matched constitutional DNA (bottom) confirms the somatic nature of this mutation. (D) Sequence electropherograms from case 32 (top) and matched constitutional DNA (bottom) demonstrate two somatic missense mutations—C121S and G128V—at similar allele frequencies.

FIG. 2: Genotypic and Clinical Data for Example 1.

FIG. 3: Comparison of clinical data based on BRAF and MAP2K1 mutation status.

FIG. 4A-C provides: (A) human wild type nucleic acid sequence for MAP2K1 (SEQ ID NO: 2); (B) wild type human ORF nucleic acid sequence for MAP2K1; (C) wild type human amino acid sequence encoded by MAP2K1 (SEQ ID NO: 1).

### DEFINITIONS

To facilitate an understanding of the present invention, a number of terms and phrases are defined below:

As used herein, the term “BRAF V600E” refers to an amino acid mutation within the BRAF protein (see, e.g., NCBI Reference Sequence: NP 004324.2 for wild type human amino acid sequence of BRAF).

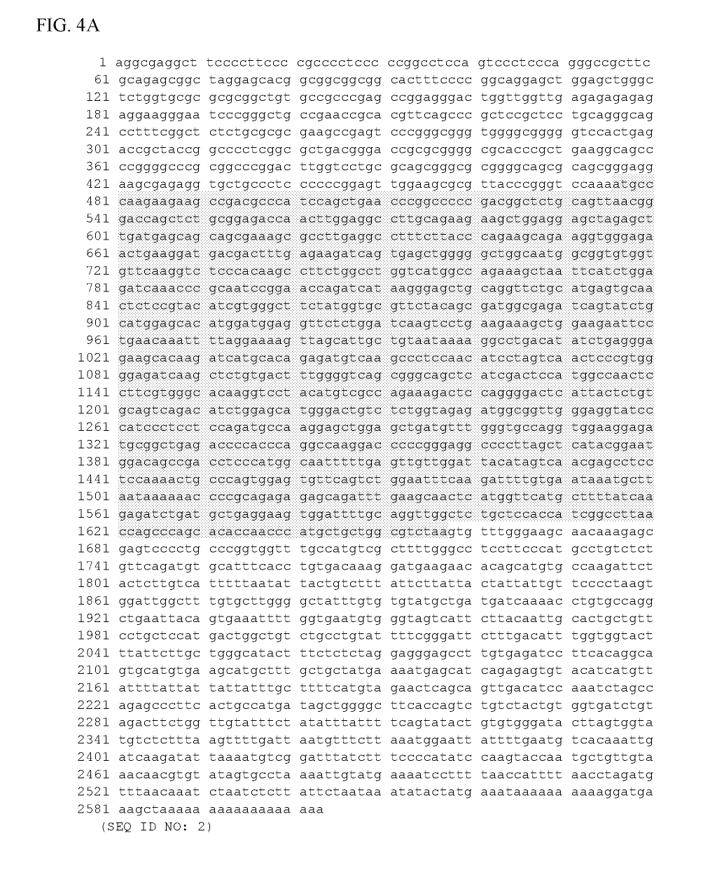

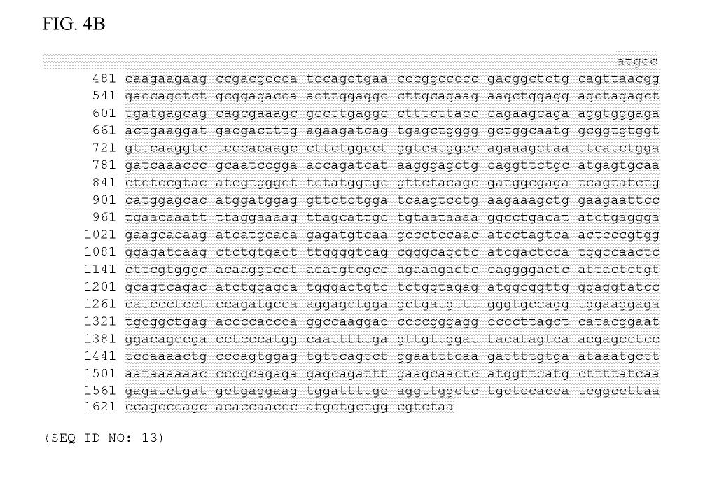

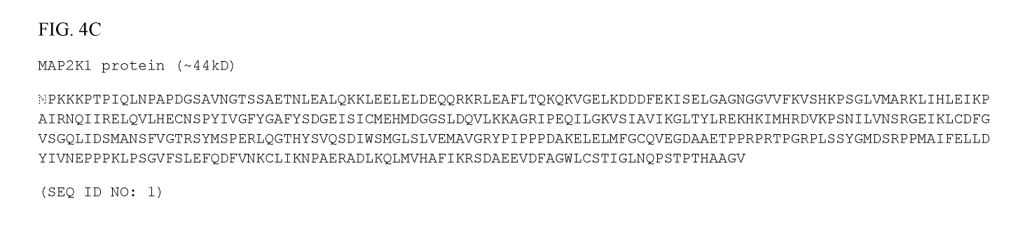

As used herein, “mitogen-activated protein kinase kinase 1” or “MAP2K1” refers to a member of the dual-specificity protein kinase family that acts as a mitogen-activated protein (MAP) kinase kinase. The MAP2K1 gene encodes the MEK1 polypeptide. An example of MAP2K1 nucleic acid and amino acid sequence is provided at NCBI Reference Sequence: NM_002755.3. FIG. 4 provides: A) human wild type nucleic acid sequence for MAP2K1 (SEQ ID NO: 2); B) wild type human ORF nucleic acid sequence for MAP2K1; C) wild type human amino acid sequence encoded by MAP2K1 (SEQ ID NO: 1).

Any of the following MEK1 (i.e., encoded by MAP2K1 gene) amino acid mutations: R47Q; R49C; F53_Q58delinsL; K57_G61del; 199_K104del; H100_I103delinsPL; E102_I103del; A106T; C121S; and G128V refer to a mutation in the MAP2K1 gene product in which the respective amino acids within the wild type sequence (SEQ ID NO: 1) are respectively altered.

Any of the following MAP2K1 nucleic acid mutations: 140G>A; 145C>T; 159_173del; 168_182del; 295_312del; 299_307delinsCTC; 303_308del; 316G>A; 304_309del; 361T>A; and 383G>T refer to a mutation in the MAP2K1 gene in which the respective nucleic acids within the wild type sequence (SEQ ID NO: 2) are respectively altered.

The term “zygosity status” as used herein means whether an individual is homozygous for a gene or gene mutation, i.e. both alleles have the same copy of a gene or gene mutation, or heterozygous for a gene or gene mutation, i.e. only one allele has a copy of the gene or gene mutation.

The term “diagnose” or “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “poor prognosis” as used herein, in the context of a patient having LCH, refers to an increased likelihood that the patient will have a worse outcome in a clinical condition relative to a patient diagnosed as having the same disease but without the mutation. A poor prognosis may be expressed in any relevant prognostic terms and may include, for example, the expectation of a reduced duration of remission, reduced survival rate, and reduced survival duration.

As used herein, the term “sample” or “biological sample” refers to any liquid or solid material obtained from a biological source, such a cell or tissue sample or bodily fluids. “Bodily fluids” include, but are not limited to, blood, serum, plasma, saliva, cerebrospinal fluid, pleural fluid, tears, lactal duct fluid, lymph, sputum, urine, saliva, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin. Exemplary sample tissues include, but are not limited to bone marrow or tissue (e.g. biopsy material).

As used herein, the term “specifically binds,” when referring to a binding moiety, is meant that the moiety is capable of discriminating between a various target sequences. For example, an oligonucleotide (e.g., a primer or probe) that specifically binds to a mutant target sequence is one that hybridizes preferentially to the target sequence (e.g., the wildtype sequence) over the other sequence variants (e.g., mutant and polymorphic sequences). Preferably, oligonucleotides specifically bind to their target sequences under high stringency hybridization conditions.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segment with a high frequency of complementary base sequences.

Exemplary hybridization conditions are as follows. High stringency generally refers to conditions that permit hybridization of only those nucleic acid sequences that form stable hybrids in 0.018M NaCl at 65° C. High stringency conditions can be provided, for example, by hybridization in 50% formamide, 5× Denhardt's solution, 5×SSC (saline sodium citrate) 0.2% SDS (sodium dodecyl sulphate) at 42° C., followed by washing in 0.1×SSC, and 0.1% SDS at 65° C. Moderate stringency refers to conditions equivalent to hybridization in 50% formamide, 5× Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5× Denhardt's solution, 6×SSC, 0.2% SDS, followed by washing in 1×SSC, 0.2% SDS, at 50° C.

As used herein, the term “biomarker” refers to an organic biomolecule which is differentially present in a sample taken from a subject of one phenotypic status (e.g. having a disease) as compared with another phenotypic status (e.g., not having the disease). A biomarker is differentially present between different phenotypic statuses if the mean or median expression level of the biomarker in the different groups is calculated to be statistically significant. In some embodiments, biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

As used herein, the term “measuring” means methods which include detecting the presence or absence of biomarker(s) in the sample, quantifying the amount of marker(s) in the sample, and/or qualifying the type of biomarker. Measuring can be accomplished by methods known in the art and those further described herein. Any suitable methods can be used to detect and measure one or more of the markers described herein.

As used herein, the term “detect” refers to identifying the presence, absence or amount of the object to be detected (e.g., a biomarker).

### DETAILED DESCRIPTION OF THE INVENTION

The present invention relates to methods and biomarkers for detection and characterization of Langerhans cell histiocytosis (LCH) in biological samples (e.g., tissue samples, blood samples, plasma samples, cell samples, serum samples). In particular, the present invention provides compositions and methods for diagnosing a patient as having a Langerhans cell histiocytosis by identifying mutations in the MAP2K1 gene or gene products.

The present invention is based on the identification of several mutations in exons 2 and 2 of the MAP2K1 gene in patients diagnosed with LCH. The mutations include, but are not limited to, the following MAP2K1 nucleic acid mutations: 140G>A; 145C>T; 159_173del; 168_182del; 295_312del; 299_307delinsCTC; 303_308del; 316G>A; 304_309del; 361T>A; and 383G>T (see, Table 2 for nucleic acid and corresponding amino acid mutations). Accordingly, the invention also provides variant nucleic acids with these gene mutations and the resulting mutated proteins, methods and reagents for the detection of the variants disclosed herein, uses of these variants for the development of detection reagents, and assays or kits that utilize such reagents.

Such mutations within the MAP2K1 gene (see, e.g., Table 2) may be assessed by any suitable method including, for example, by nucleic acid sequencing or oligonucleotide hybridization. For example, such mutations may be assessed by amplifying a target sequence of a MAP2K1 nucleic acid (e.g., genomic DNA, RNA, or cDNA) containing all or a portion of the mutation. Relatedly, detection may involve using probes and/or primers capable of specifically hybridizing to the mutation site. Target sequences (including primer and probe sequences encompassing this mutation) may be of any suitable length (e.g., 20, 25, 30, 35, 40, 50, 100, 200, 300, or more nucleotides in length).

Alternatively, such mutations within the MAP2K1 gene (see, e.g., Table 2) may be assessed by evaluating the MEK1 protein (encoded by MAP2K1 gene) present in a patient sample such as by specifically detecting a protein variant. MEK1 protein assessment may be performed by any appropriate method including amino acid sequencing or through the use of mutant MEK1-specific antibodies (e.g., using an ELISA). Mutant MEK1 proteins may be assessed by amino acid sequencing of all or a portion of the MEK1 protein comprising the amino acid sequence encoded by one or more MAP2K1 nucleic acid mutations (e.g., 140G>A; 145C>T; 159_173del; 168_182del; 295_312del; 299_307delinsCTC; 303_308del; 316G>A; 304_309del; 361T>A; and 383G>T) (e.g., the corresponding amino acid sequence encoded by the MAP2K1 nucleic acid mutations: R47Q; R49C; F53_Q58delinsL; K57_G61del; 199_K104del; H100_I103delinsPL; E102_I103del; A106T; C121S; and G128V). Optionally, antibodies (polyclonal or monoclonal) can be raised against the a polypeptide epitope having the amino acid sequence encoded by one or more of such MAP2K1 gene mutations.

The methods and compositions of this invention may be used to detect mutations in the MAP2K1 gene using a biological sample obtained from an individual. The nucleic acid (DNA or RNA) may be isolated from the sample according to any methods well known to those of skill in the art. Examples include tissue samples or any cell-containing or acellular bodily fluid. Biological samples may be obtained by standard procedures and may be used immediately or stored, under conditions appropriate for the type of biological sample, for later use.

Methods of obtaining test samples are well known to those of skill in the art and include, but are not limited to, aspirations, tissue sections, drawing of blood or other fluids, surgical or needle biopsies, and the like. The test sample may be obtained from an individual or patient diagnosed as having LCH. The test sample may be a cell-containing liquid or a tissue. Samples may include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the individual's cells to detect using polymerase chain reaction.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70° C. until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood can be drawn by standard methods into a collection tube, typically siliconized glass, either without anticoagulant for preparation of serum, or with EDTA, sodium citrate, heparin, or similar anticoagulants for preparation of plasma. A requirement for preparing plasma or serum for storage, although not an absolute requirement, is that plasma or serum is first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparanase, followed by removal of calcium prior to reverse transcription (see, e.g., Imai, H., et al., J. Virol. Methods 36:181-184, (1992)). Thus, EDTA is a suitable anticoagulant for blood specimens in which PCR amplification is planned.

Variant MAP2K1 nucleic acids or polypeptides (MEK1 polypeptides) of the present invention may be detected as genomic DNA or mRNA using a variety of nucleic acid techniques known to those of ordinary skill in the art, including but not limited to: nucleic acid sequencing; nucleic acid hybridization; and, nucleic acid amplification.

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, fluorescent or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

Some embodiments of the present invention utilize next generation or high-throughput sequencing. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some embodiments, sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (see, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and fluorescent acceptor molecules, resulting in detectable fluorescence resonance energy transfer (FRET) upon nucleotide addition.

In some embodiments, capillary electrophoresis (CE) is utilized to analyze amplification fragments. During capillary electrophoresis, nucleic acids (e.g., the products of a PCR reaction) are injected electrokinetically into capillaries filled with polymer. High voltage is applied so that the fluorescent DNA fragments are separated by size and are detected by a laser/camera system. In some embodiments, CE systems from Life Technogies (Grand Island, N.Y.) are utilized for fragment sizing (see e.g., U.S. Pat. No. 6,706,162, U.S. Pat. No. 8,043,493, each of which is herein incorporated by reference in its entirety).

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, the present invention provides nucleic acid probes specific for a particular MAP2K1 variant. For example, in some embodiments, separate nucleic acid probes are provided that are only specific for one MAP2K1 variant as described herein (see, e.g., MAP2K1 mutations shown in Table 2). In some embodiments, such separate nucleic acid probes specific for a MAP2K1 variant will not bind the respective wild type equivalent.

In some embodiments, such separate nucleic acid probes specific for a MAP2K1 variant will not bind different MAP2K1 variants.

In some embodiments, microarrays are utilized for detection of MAP2K1 nucleic acid sequences and MEK1 amino acid sequences. Examples of microarrays include, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Arrays can also be used to detect copy number variations at a specific locus. These genomic microarrays detect microscopic deletions or other variants that lead to disease causing alleles.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

The nucleic acid to be amplified may be from a biological sample such as an organism, cell culture, tissue sample, and the like. The biological sample can be from a subject which includes any animal, preferably a mammal. A preferred subject is a human, which may be a patient presenting to a medical provider for diagnosis or treatment of a disease (e.g., LCH). The volume of plasma or serum used in the extraction may be varied dependent upon clinical intent, but volumes of 100 μL to one milliliter of plasma or serum are usually sufficient.

Various methods of extraction are suitable for isolating the DNA or RNA. Suitable methods include phenol and chloroform extraction (see, e.g., Maniatis et al., Molecular Cloning, A Laboratory Manual, 2d, Cold Spring Harbor Laboratory Press, page 16.54 (1989)). Numerous commercial kits also yield suitable DNA and RNA including, but not limited to, QIAamp™ mini blood kit, Agencourt Genfind™, Roche Cobas® Roche MagNA Pure® or phenol:chloroform extraction using Eppendorf Phase Lock Gels®, and the NucliSens extraction kit (Biomerieux, Marcy l'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science, Indianapolis, Ind.).

Nucleic acid extracted from tissues, cells, plasma or serum can be amplified using nucleic acid amplification techniques well known in the art. Many of these amplification methods can also be used to detect the presence of mutations simply by designing oligonucleotide primers or probes to interact with or hybridize to a particular target sequence in a specific manner. By way of example, but not by way of limitation, these techniques can include the polymerase chain reaction (PCR), reverse transcriptase polymerase chain reaction (RT-PCR), nested PCR, ligase chain reaction (see, e.g., Abravaya, K., et al., Nucleic Acids Research, 23:675-682, (1995)), branched DNA signal amplification (see, e.g., Urdea, M. S., et al., AIDS, 7 (suppl 2):S11-514, (1993)), amplifiable RNA reporters, Q-beta replication, transcription-based amplification, boomerang DNA amplification, strand displacement activation, cycling probe technology, isothermal nucleic acid sequence based amplification (NASBA) (see, e.g., Kievits, T. et al., J Virological Methods, 35:273-286, (1991)), Invader Technology, or other sequence replication assays or signal amplification assays. These methods of amplification each described briefly below and are well-known in the art.

Some methods employ reverse transcription of RNA to cDNA. As noted, the method of reverse transcription and amplification may be performed by previously published or recommended procedures, which referenced publications are incorporated herein by reference in their entirety. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011) (see, e.g., Rashtchian, A., PCR Methods Applic., 4:S83-S91, (1994)).

PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al., J of Clin Micro, 33(3):556-561 (1995). Briefly, a PCR reaction mixture includes two specific primers, dNTPs, approximately 0.25 U of Taq polymerase, and 1× PCR Buffer.

LCR is a method of DNA amplification similar to PCR, except that it uses four primers instead of two and uses the enzyme ligase to ligate or join two segments of DNA. LCR can be performed as according to Moore et al., J Clin Micro, 36(4):1028-1031 (1998). Briefly, an LCR reaction mixture contains two pair of primers, dNTP, DNA ligase and DNA polymerase representing about 90 to which is added 100 μl of isolated nucleic acid from the target organism. Amplification is performed in a thermal cycler (e.g., LCx of Abbott Labs, Chicago, Ill.).

TAS is a system of nucleic acid amplification in which each cycle is comprised of a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS, 86:1173-7 (1989). Briefly, extracted RNA is combined with TAS amplification buffer and bovine serum albumin, dNTPs, NTPs, and two oligonucleotide primers, one of which contains a PBS. The sample is heated to denature the RNA template and cooled to the primer annealing temperature. Reverse transcriptase (RT) is added the sample incubated at the appropriate temperature to allow cDNA elongation. Subsequently T7 RNA polymerase is added and the sample is incubated at 37° C. for approximately 25 minutes for the synthesis of RNA. The above steps are then repeated. Alternatively, after the initial cDNA synthesis, both RT and RNA polymerase are added following a 1 minute 100° C. denaturation followed by an RNA elongation of approximately 30 minutes at 37° C. TAS can be also be performed on solid phase as according to Wylie et al., J Clin Micro, 36(12):3488-3491 (1998). In this method, nucleic acid targets are captured with magnetic beads containing specific capture primers. The beads with captured targets are washed and pelleted before adding amplification reagents which contains amplification primers, dNTP, NTP, 2500 U of reverse transcriptase and 2500 U of T7 RNA polymerase. A 100 μA TMA reaction mixture is placed in a tube, 200 μA oil reagent is added and amplification is accomplished by incubation at 42° C. in a waterbath for one hour.

NASBA is a transcription-based amplification method which amplifies RNA from either an RNA or DNA target. NASBA is a method used for the continuous amplification of nucleic acids in a single mixture at one temperature. For example, for RNA amplification, avian myeloblastosis virus (AMV) reverse transcriptase, RNase H and T7 RNA polymerase are used. This method can be performed as according to Heim, et al., Nucleic Acids Res., 26(9):2250-2251 (1998). Briefly, an NASBA reaction mixture contains two specific primers, dNTP, NTP, 6.4 U of AMV reverse transcriptase, 0.08 U of Escherichia coli Rnase H, and 32 U of T7 RNA polymerase. The amplification is carried out for 120 min at 41° C. in a total volume of 20 μl.

In a related method, self-sustained sequence-replication (3SR) reaction, isothermal amplification of target DNA or RNA sequences in vitro using three enzymatic activities: reverse transcriptase, DNA-dependent RNA polymerase and Escherichia coli ribonuclease H. This method may be modified from a 3-enzyme system to a 2-enzyme system by using human immunodeficiency virus (HIV)-1 reverse transcriptase instead of avian myeloblastosis virus (AMV) reverse transcriptase to allow amplification with T7 RNA polymerase but without E. coli ribonuclease H. In the 2-enzyme 3SR, the amplified RNA is obtained in a purer form compared with the 3-enzyme 3SR (Gebinoga & Oehlenschlager Eur J Biochem, 235:256-261, 1996).

SDA is an isothermal nucleic acid amplification method. A primer containing a restriction site is annealed to the template. Amplification primers are then annealed to 5′ adjacent sequences (forming a nick) and amplification is started at a fixed temperature. Newly synthesized DNA strands are nicked by a restriction enzyme and the polymerase amplification begins again, displacing the newly synthesized strands. SDA can be performed as according to Walker, et al., PNAS, 89:392-6 (1992). Briefly, an SDA reaction mixture contains four SDA primers, dGTP, dCTP, TTP, dATP, 150 U of Hinc II, and 5 U of exonuclease-deficient of the large fragment of E. coli DNA polymerase I (exo-Klenow polymerase). The sample mixture is heated 95° C. for 4 minutes to denature target DNA prior to addition of the enzymes. After addition of the two enzymes, amplification is carried out for 120 min. at 37° C. in a total volume of 50 μl. Then, the reaction is terminated by heating for 2 min. at 95° C.

The Q-beta replication system uses RNA as a template. Q-beta replicase synthesizes the single-stranded RNA genome of the coliphage Qβ. Cleaving the RNA and ligating in a nucleic acid of interest allows the replication of that sequence when the RNA is replicated by Q-beta replicase (Kramer & Lizardi Trends Biotechnol. 1991 9(2):53-8, 1991). A variety of amplification enzymes are well known in the art and include, for example, DNA polymerase, RNA polymerase, reverse transcriptase, Q-beta replicase, thermostable DNA and RNA polymerases. Because these and other amplification reactions are catalyzed by enzymes, in a single step assay the nucleic acid releasing reagents and the detection reagents should not be potential inhibitors of amplification enzymes if the ultimate detection is to be amplification based. Amplification methods suitable for use with the present methods include, for example, strand displacement amplification, rolling circle amplification, primer extension preamplification, or degenerate oligonucleotide PCR (DOP). These methods of amplification are well known in the art and each described briefly below.

In suitable embodiments, PCR is used to amplify a target or marker sequence of interest. The skilled artisan is capable of designing and preparing primers that are appropriate for amplifying a target or marker sequence. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill. For example, the length of a short nucleic acid or oligonucleotide can relate to its hybridization specificity or selectivity.

For analyzing mutations and other variant nucleic acids, it may be appropriate to use oligonucleotides specific for alternative alleles. Such oligonucleotides which detect single nucleotide variations in target sequences may be referred to by such terms as “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature, 324:163-166 (1986); Dattagupta, EP235,726; and Saiki, WO 89/11548. In one embodiment, a probe or primer may be designed to hybridize to a segment of target DNA such that the SNP aligns with either the 5′ most end or the 3′ most end of the probe or primer.

In some embodiments, the amplification may include a labeled primer, thereby allowing detection of the amplification product of that primer. In particular embodiments, the amplification may include a multiplicity of labeled primers; typically, such primers are distinguishably labeled, allowing the simultaneous detection of multiple amplification products.

In one type of PCR-based assay, an allele-specific primer hybridizes to a region on a target nucleic acid molecule that overlaps a SNP position and only primes amplification of an allelic form to which the primer exhibits perfect complementarity (Gibbs, 1989, Nucleic Acid Res., 17:2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target mutation position) because this position is most destabilizing to elongation from the primer (see, e.g., WO 93/22456).

In a specific embodiment, a primer contains a sequence substantially complementary to a segment of a target mutation-containing nucleic acid molecule except that the primer has a mismatched nucleotide in one of the three nucleotide positions at the 3′-most end of the primer, such that the mismatched nucleotide does not base pair with a particular allele at the mutation site. In one embodiment, the mismatched nucleotide in the primer is the second from the last nucleotide at the 3′-most position of the primer. In another embodiment, the mismatched nucleotide in the primer is the last nucleotide at the 3′-most position of the primer.

In one embodiment, primer or probe is labeled with a fluorogenic reporter dye that emits a detectable signal. While a suitable reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

The present invention also contemplates reagents that do not contain (or that are complementary to) a mutated nucleotide sequence identified herein but that are used to assay one or more of the mutations disclosed herein. For example, primers that flank, but do not hybridize directly to a target position provided herein are useful in primer extension reactions in which the primers hybridize to a region adjacent to the target position (i.e., within one or more nucleotides from the target mutation site). During the primer extension reaction, a primer is typically not able to extend past a target mutation site if a particular nucleotide (allele) is present at that target site, and the primer extension product can readily be detected in order to determine which allele (i.e., wildtype or mutant) is present. For example, particular ddNTPs are typically used in the primer extension reaction to terminate primer extension once a ddNTP is incorporated into the extension product. Thus, reagents that bind to a nucleic acid molecule in a region adjacent to a mutation site, even though the bound sequences do not necessarily include the mutation site itself, are also encompassed by the present invention.

Variant nucleic acids may be amplified prior to detection or may be detected directly during an amplification step (i.e., “real-time” methods). In some embodiments, the target sequence is amplified and the resulting amplicon is detected by electrophoresis. In some embodiments, the specific mutation or variant is detected by sequencing the amplified nucleic acid. In some embodiments, the target sequence is amplified using a labeled primer such that the resulting amplicon is detectably labeled. In some embodiments, the primer is fluorescently labeled.

In one embodiment, detection of a variant nucleic acid is performed using the TaqMan® assay, which is also known as the 5′ nuclease assay (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

TaqMan® primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present invention are useful in diagnostic assays for neurodevelopmental disorders and related pathologies, and can be readily incorporated into a kit format. The present invention also includes modifications of the TaqMan® assay well known in the art such as the use of Molecular Beacon probes (U.S. Pat. Nos. 5,118,801 and 5,312,728) and other variant formats (U.S. Pat. Nos. 5,866,336 and 6,117,635).

In an illustrative embodiment, real time PCR is performed using TaqMan® probes in combination with a suitable amplification/analyzer such as the ABI Prism® 7900HT Sequence Detection System. The ABI PRISM® 7900HT Sequence Detection System is a high-throughput real-time PCR system that detects and quantitates nucleic acid sequences. Briefly, TaqMan® probes specific for the amplified target or marker sequence are included in the PCR amplification reaction. These probes contain a reporter dye at the 5′ end and a quencher dye at the 3′ end. Probes hybridizing to different target or marker sequences are conjugated with a different fluorescent reporter dye. During PCR, the fluorescently labeled probes bind specifically to their respective target or marker sequences; the 5′ nuclease activity of Taq polymerase cleaves the reporter dye from the probe and a fluorescent signal is generated. The increase in fluorescence signal is detected only if the target or marker sequence is complementary to the probe and is amplified during PCR. A mismatch between probe and target greatly reduces the efficiency of probe hybridization and cleavage. The ABI Prism 7700HT or 7900HT Sequence detection System measures the increase in fluorescence during PCR thermal cycling, providing “real time” detection of PCR product accumulation. Real time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually.

Other methods of probe hybridization detected in real time can be used for detecting amplification a target or marker sequence flanking a tandem repeat region. For example, the commercially available MGB Eclipse™ probes (Epoch Biosciences), which do not rely on a probe degradation can be used. MGB Eclipse™ probes work by a hybridization-triggered fluorescence mechanism. MGB Eclipse™ probes have the Eclipse™ Dark Quencher and the MGB positioned at the 5′-end of the probe. The fluorophore is located on the 3′-end of the probe. When the probe is in solution and not hybridized, the three dimensional conformation brings the quencher into close proximity of the fluorophore, and the fluorescence is quenched. However, when the probe anneals to a target or marker sequence, the probe is unfolded, the quencher is moved from the fluorophore, and the resultant fluorescence can be detected.

Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled. Amplified fragments may be detected using standard gel electrophoresis methods. For example, in preferred embodiments, amplified fractions are separated on an agarose gel and stained with ethidium bromide by methods known in the art to detect amplified fragments.

Another suitable detection methodology involves the design and use of bipartite primer/probe combinations such as Scorpion™ probes. These probes perform sequence-specific priming and PCR product detection is achieved using a single molecule. Scorpion™ probes comprise a 3′ primer with a 5′ extended probe tail comprising a hairpin structure which possesses a fluorophore/quencher pair. The probe tail is “protected” from replication in the 5′ to 3′ direction by the inclusion of hexethlyene glycol (HEG) which blocks the polymerase from replicating the probe. The fluorophore is attached to the 5′ end and is quenched by a moiety coupled to the 3′ end. After extension of the Scorpion™ primer, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion™, resulting in an extension product. A fluorescent signal is generated due to the separation of the fluorophore from the quencher resulting from the binding of the probe element of the Scorpion™ to the extension product. Such probes are described in Whitcombe et al., Nature Biotech 17: 804-807 (1999).

In other embodiments, variant MEK1 polypeptides (encoded by the MAP2K1 gene) are detected. Any suitable method may be used to detect truncated or mutant MEK1 polypeptides. For example, detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

MEK1 proteins (encoded by the MAP2K1 gene) with and without insertion/truncation mutation may be recovered from biological sample from an individual, culture medium or from host cell lysates. If membrane-bound, it can be released from the membrane using a suitable detergent solution (e.g., Triton-X 100) or by enzymatic cleavage. Cells employed in the expression of MEK1 protein can be disrupted by various physical or chemical means, such as freeze-thaw cycling, sonication, mechanical disruption, or cell lysing agents.

It may be desired to purify MEK1 protein from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the BCR-ABL. Various methods of protein purification may be employed and such methods are known in the art and described for example in Deutscher, Methods in Enzymology (1990), 182:83-89; Scopes, Protein Purification: Principles and Practice, Springer-Verlag, New York (1982). The purification step(s) selected will depend, for example, on the nature of the production process, source of MEK1 used and the particular MEK1 produced.

Several methods for detection of proteins are well known in the art. Detection of the proteins could be by resolution of the proteins by SDS polyacrylamide gel electrophoresis (SDS PAGE), followed by staining the proteins with suitable stain for example, Coomassie Blue. The MEK1 proteins with and without a mutation (encoded by the MAP2K1 gene) can be differentiated from each other and also from other proteins by Western blot analysis using mutation-specific antibodies. Methods of Western blot are well known in the art and described for example in W. Burnette W. N. Anal. Biochem. 1981; 112 (2): 195-203.

Alternatively, flow cytometry may be applied to detect the mutant and wildtype MEK1 protein. Antibodies specific for either the mutant or wildtype protein can be coupled to beads and can be used in the flow cytometry analysis.

In some embodiments, protein microarrays may be applied to identify the various MEK1 protein variants. Methods of protein arrays are well known in the art. In one example, antibodies specific for each protein may be immobilized on the solid surface such as glass or nylon membrane. The proteins can then be immobilized on the solid surface through the binding of the specific antibodies. Antibodies may be applied that bind specifically to a second epitope (e.g., an epitope common to the mutant and wildtype) of the MEK1 proteins. The first antibody/protein/second antibody complex can then be detected using a detectably labeled secondary antibody. The detectable label can be detected as discussed for polynucleotides.

Various procedures known in the art may be used for the production of antibodies to epitopes of the MEK1 protein that may be used to distinguish among the protein variants. Such antibodies include but are not limited to polyclonal, monoclonal, chimeric, single chain, Fab fragments and fragments produced by a Fab expression library.

Antibodies may be radioactively labeled allowing one to follow their location and distribution in the body after injection. Radioactivity tagged antibodies may be used as a non-invasive diagnostic tool for imaging de novo cells of tumors and metastases.

Immunotoxins may also be designed which target cytotoxic agents to specific sites in the body. For example, high affinity MEK1-specific monoclonal antibodies may be covalently complexed to bacterial or plant toxins, such as diphtheria toxin, abrin or ricin. A general method of preparation of antibody/hybrid molecules may involve use of thiol-crosslinking reagents such as SPDP, which attack the primary amino groups on the antibody and by disulfide exchange, attach the toxin to the antibody. The hybrid antibodies may be used to specifically eliminate mutant MEK1 protein-expressing cells.

For the production of antibodies, various host animals may be immunized by injection with the full length or fragment of MEK1 proteins including but not limited to rabbits, mice, rats, etc. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to MEK1 proteins may be prepared by using any technique which provides for the production of antibody molecules by continuous cell lines in culture. These include but are not limited to the hybridoma technique originally described by Kohler and Milstein, (Nature (1975), 256:495-497), the human B-cell hybridoma technique (Kosbor et al., Immunology Today (1983), 4:72; Cote et al. Proc. Natl. Acad. Sci. (1983), 80:2026-2030) and the EBV-hybridoma technique (Cole et al., Monoclonal Antibodies and Cancer Therapy (1985), Alan R. Liss, Inc., pp. 77-96). In addition, techniques developed for the production of “chimeric antibodies” (Morrison et al., Proc. Natl. Acad. Sci. USA (1984), 81:6851-6855; Neuberger et al., Nature (1984), 312:604-608; Takeda et al., Nature (1985), 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce MEK1 protein-specific single chain antibodies.

Antibody fragments may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., Science. 1989; 246: 1275-1281) to allow rapid and easy identification of monoclonal Fab fragments with the desired specificity.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology for detecting a specific MEK1 variant described herein (e.g., a MEK1 variant encoded by one of the MAP2K1 mutations described herein) combines mass spectrometry with immunoassay. First, a biospecific capture reagent (e.g., an antibody, aptamer or Affibody that recognizes the biomarker and other forms of it) is used to capture the biomarker of interest (e.g., a MEK1 variant). Preferably, the biospecific capture reagent is bound to a solid phase, such as a bead, a plate, a membrane or an array. After unbound materials are washed away, the captured analytes are detected and/or measured by mass spectrometry. In some embodiments, such methods also permit capture of protein interactors, if present, that are bound to the proteins or that are otherwise recognized by antibodies and that, themselves, can be biomarkers. Various forms of mass spectrometry are useful for detecting the protein forms, including laser desorption approaches, such as traditional MALDI or SELDI, and electrospray ionization.

In some embodiments, a biomarker of this invention (e.g., a MEK1 variant encoded by one of MAP2K1 mutations described herein) is detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In some embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In some embodiments, the mass spectrometric technique for use is “Surface Enhanced Laser Desorption and Ionization” or “SELDI,” as described, for example, in U.S. Pat. No. 5,719,060 and No. 6,225,047; each herein incorporated by reference in its entirety. This refers to a method of desorption/ionization gas phase ion spectrometry (e.g. mass spectrometry) in which an analyte (e.g., one or more of the biomarkers of the present invention) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047; herein incorporated by reference in its entirety. A “bioselective adsorbent” refers to an adsorbent that binds to an analyte with an affinity of at least 10⁻⁸M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, MAC-30 and IMAC 40 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding. Such biochips are further described in: U.S. Pat. Nos. 7,045,366, 6,579,719; 6,897,072; 6,555,813; U.S. Patent Publication Nos. U.S. 2003-0032043; US 2003-0218130; and PCT International Publication No. WO 03/040700; each herein incorporated by reference in its entirety.

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate “C18 SEND”). SEND is further described in U.S. Pat. No. 6,124,137 and PCT International Publication No. WO 03/64594; each herein incorporated in its entirety.

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

In some embodiments, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. For example, in some embodiments, the present invention provides biochips having attached thereon one or more capture reagents specific for a MAP2K1 variant of the present invention.

Protein biochips are biochips adapted for the capture of polypeptides (e.g., a MEK1 variant encoded by one of MAP2K1 mutations described herein). Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047, 6,537,749, 6,329,209, and 5,242,828, and PCT International Publication Nos. WO 00/56934, and WO 03/048768; each herein incorporated by reference in its entirety.

In certain embodiments, the present invention provides methods for managing a subject's treatment based on the status (e.g., presence or absence of LCH). Such management includes the actions of the physician or clinician subsequent to determining LCH status. For example, if a physician makes a diagnosis of LCH, then a certain regime of treatment, such as prescription or administration of therapeutic agent might follow. Alternatively, a diagnosis of non-mature LCH might be followed with further testing to determine a specific disease that the patient might be suffering from. Also, if the diagnostic test gives an inconclusive result on LCH status, further tests may be called for.

In another aspect, the present invention provides compositions of matter based on the biomarkers of this invention. For example, in one embodiment, the present invention provides a biomarker of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. For example, in some embodiments, the present invention provides compositions comprising a purified MEK1 variant (e.g., a MEK1 variant encoded by one of MAP2K1 mutations described herein).

Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, the present invention provides a biospecific capture reagent, optionally in purified form, that specifically binds a biomarker of this invention. In one embodiment, the biospecific capture reagent is an antibody. Such compositions are useful for detecting the biomarker in a detection assay, e.g., for diagnostics.

In another embodiment, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect the present invention provides a composition comprising a biospecific capture reagent, such as an antibody, bound to a biomarker of this invention, the composition optionally being in purified form. Such compositions are useful for purifying the biomarker or in assays for detecting the biomarker.

In another embodiment, this invention provides an article comprising a solid substrate to which is attached an adsorbent, e.g., a chromatographic adsorbent or a biospecific capture reagent, to which is further bound a biomarker of this invention.

In another embodiment, the invention provides compositions comprising reaction mixtures formed through, for example, binding of a biomarker of the present invention with a detection marker (e.g., antibody, proble, biochip, etc.) (e.g., via a detection assay of the present invention). In some embodiments, “reaction mixture” comprises any material sufficient, necessary, or useful for conducting any of the assays described herein. In some embodiments, the present invention provides compositions comprising reaction mixtures comprising extension products complementary to a specific mutation. In some embodiments, the present invention provides compositions comprising reaction mixtures comprising extension products complementary to a specific mutation and sequences immediately surrounding such a mutation. In some embodiments, the extension product has thereon an labeling agent (e.g., a fluorophore or other label). In some embodiments, the present invention provides compositions comprising reaction mixtures comprising extension products complementary to a specific mutation bound with such a complementary sequence. In some embodiments, the present invention provides compositions comprising reaction mixtures comprising extension products complementary to a specific mutation bound with such a complementary sequence, wherein the binding is to a solid surface, a biochip (e.g., in single copy or multiple copies). In some embodiments, the present invention provides compositions comprising fragments of a peptide of interest. In some embodiments, the present invention provides compositions comprising a peptide of interest in a mass-spectrometry compatible buffer.

In some embodiments, a computer-based analysis program is used to translate raw data generated by detection assay (e.g., the presence, absence, or amount of a given MAP2K1 related allele or MEK1 polypeptide) of the present invention into data of predictive value for a clinician. The clinician can access the predictive data using any suitable means. Thus, in some preferred embodiments, the present invention provides the further benefit that the clinician, who may not be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence of wild type or mutant MAP2K1 related allele or MEK1 protein), specific for the screening, diagnostic or prognostic information desired for the subject.

The profile data is then prepared in a format suitable for interpretation by a treating clinician. For example, rather than providing raw data, the prepared format may represent a diagnosis or risk assessment (e.g., diagnosis or prognosis of LCH) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counselling based on the results. In some embodiments, the data is used for research use. For example, the data may be used to further optimize the inclusion or elimination of markers as useful indicators of a particular condition or stage of disease.

In some embodiments, the methods disclosed herein are useful in monitoring the treatment of LCH. For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of LCH-specific marker detection results in a sample obtained from a subject to, for example, a library of such marker patterns known to be indicative of the presence or absence of LCH, or a particular stage or prognosis of LCH.

In some embodiments, the present invention provides computer programming for analyzing and comparing a first and a second pattern of LCH-specific marker detection results from a sample taken at least two different time points. In some embodiments, the first pattern may be indicative of a pre-cancerous condition and/or low risk condition for LCH and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of LCH-specific marker detection results from a sample to a library of LCH-specific marker patterns known to be indicative of the presence or absence of LCH, wherein the comparing provides, for example, a differential diagnosis between an aggressively malignant LCH and a less aggressive LCH (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of gastrointestinal neoplasm-specific marker detection results from a sample). This information received can be stored at least temporarily in a database, and data analyzed in comparison to a library of marker patterns known to be indicative of the presence or absence of a pre-cancerous condition, or known to be indicative of a stage and/or grade of gastrointestinal cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of LCH-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a sample obtained from a subject is to be input by a user (e.g., a technician or someone performing the assays)) and transmitted to a remote site to a second computer processor for analysis (e.g., where the pattern of LCH-specific marker detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of LCH, or monitoring the progression of a pre-cancerous condition or LCH. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of LCH, or a relational database (RDB) which can include data input by the user and data output. The computer processors can be processors that are typically found in personal desktop computers (e.g., IBM, Dell, Macintosh), portable computers, mainframes, minicomputers, tablet computer, smart phone, or other computing devices.

The input components can be complete, stand-alone personal computers offering a full range of power and features to run applications. The user component usually operates under any desired operating system and includes a communication element (e.g., a modem or other hardware for connecting to a network using a cellular phone network, Wi-Fi, Bluetooth, Ethernet, etc.), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing LCH or having an aggressive form of LCH. In some embodiments, such methods involve obtaining a blood or blood product sample from a subject (e.g., a human at risk for developing LCH; a human undergoing a routine physical examination, or a human diagnosed with LCH), detecting the presence or absence of MAP2K1 variants described herein (see, e.g., MAP2K1 mutations shown in Table 2) in the sample, and generating a risk profile for developing LCH or progressing to a metastatic or aggressive form of such LCH. For example, in some embodiments, a generated profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers as determined with the methods of the present invention. In some embodiments, the presence and absence of specific MAP2K1 variants described herein (see, e.g., MAP2K1 mutations shown in Table 2) as determined with the methods of the present invention are imputed into such an algorithm, and the risk profile is reported based upon a comparison of such input with established norms (e.g., established norm for pre-cancerous condition, established norm for various risk levels for developing LCH, established norm for subjects diagnosed with various stages of LCH). In some embodiments, the risk profile indicates a subject's risk for developing LCH or a subject's risk for re-developing LCH. In some embodiments, the risk profile indicates a subject to be, for example, a very low, a low, a moderate, a high, and a very high chance of developing or re-developing LCH or having a poor prognosis (e.g., likelihood of long term survival) from LCH. In some embodiments, a health care provider (e.g., an oncologist) will use such a risk profile in determining a course of treatment or intervention (e.g., biopsy, wait and see, referral to an oncologist, referral to a surgeon, etc.).

The present inventions also contemplate diagnostic systems in kit form. A diagnostic system of the present inventions may include a kit which contains, in an amount sufficient for at least one assay, any of the hybridization assay probes, amplification primers, and/or antibodies against MEK1 wild type and mutant proteins in a packaging material. Typically, the kits will also include instructions recorded in a tangible form (e.g., contained on paper or an electronic medium) for using the packaged probes, primers, and/or antibodies in a detection assay for determining the presence or amount of variant mRNA or protein in a test sample.

The various components of the diagnostic systems may be provided in a variety of forms. For example, the required enzymes, the nucleotide triphosphates, the probes, primers, and/or antibodies may be provided as a lyophilized reagent. These lyophilized reagents may be pre-mixed before lyophilization so that when reconstituted they form a complete mixture with the proper ratio of each of the components ready for use in the assay. In addition, the diagnostic systems of the present inventions may contain a reconstitution reagent for reconstituting the lyophilized reagents of the kit. In preferred kits, the enzymes, nucleotide triphosphates and required cofactors for the enzymes are provided as a single lyophilized reagent that, when reconstituted, forms a proper reagent for use in the present amplification methods.

Some preferred kits may further contain a solid support for anchoring a nucleic acid of interest (e.g., MAP2K1 nucleic acid) on the solid support. The target nucleic acid may be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces may comprise a first member of a binding pair and the capture probe or the target nucleic acid may comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, antigen/antibody.

### Experimental

The following examples are provided in order to demonstrate and further illustrate certain preferred embodiments and aspects of the present invention and are not to be construed as limiting the scope thereof.

### EXAMPLE I

Cases of Langerhans cell histiocytosis within the archives of the Department of Pathology at the University of Michigan with institutional review board approval were studied. DNA was extracted from available non-decalcified, formalin-fixed paraffin-embedded samples in which at least 30% neoplastic nuclei could be isolated using the Pinpoint Slide DNA Isolation System (Zymo Research). To discover genetic mechanisms that might explain ERK1 activation in the absence of BRAF V600E, 8 LCH cases were initially screened using both BRAF V600E allele-specific PCR (see, e.g., Brown N A, et al., Immunohistochem Mol Morphol. Prepublished on Feb. 5, 2014, as DOI 10-1097/PAI.0000000000000024) and the Ion AmpliSeq Comprehensive Cancer Panel. Allele-specific PCR was performed.

For each of the 8 cases, sequencing libraries were generated using the Ion AmpliSeq Comprehensive Cancer Panel (Life Technologies). Approximately 40 ng of starting DNA from each sample block was amplified (10 ng per primer pool). Libraries were barcoded (IonXpress Barcode Kit, Life Technologies) and equalized (Ion Library Equalizer Kit) to a final concentration of approximately 100 pM. Emulsification PCR was performed using the One Touch DL instrument and template-positive Ion Sphere particles were enriched using the OneTouch ES instrument according to the manufacturer's instructions. Sequencing was performed on a 318 chip on the Ion Torrent PGM following the recommended protocol. Reads were aligned to hg19 and variants were called using the Torrent Suite 4.0.2.

Upon identification of a MAP2K1 mutation within exon 3 in one of 8 cases using the Ion AmpliSeq Comprehensive Cancer panel, all eight cases were evaluated using bidirectional Sanger sequencing of MAP2K1 exons 2 and 3. An additional 32 cases of LCH were then evaluated using BRAF V600E allele-specific PCR and MAP2K1 exon 2 and 3 bidirectional Sanger sequencing. DNA was sequenced using the BigDye Terminator V1.1 sequencing kit (Applied Biosystems) and the 3130×1 DNA Analyzer (Applied Biosystems). Primers are listed in Table 1. Sanger sequencing of exon 15 of the BRAF gene was performed for one case with a BRAF insertion mutation (see, e.g., Hookim K, Cancer Cytopathol. 2012; 120(1):52-61).

TABLE 1
MAP2K1 primers used for Sanger sequencing

| Target | Forward | Reverse |
|---|---|---|
| MAP2K1 | TGACTTGTGCTCCCCACTTT | GTCCCCAGGCTTCTAAGT |
| exon 2 | (SEQ ID NO: 12) | ACC |
| PCR | | (SEQ ID NO: 14) |
| MAP2K1 | GTGCTCCCCACTTTGGAAC | CCAGGCTTCTAAGTACCC |
| exon 2 | (SEQ ID NO: 3) | TGAG |
| Sequenc- | | (SEQ ID NO: 4) |
| ing | | |
| MAP2K1 | TCATCCCTTCCTCCCTCTTT | CTCTTAAGGCCATTGCTC |
| exon 3 | (SEQ ID NO: 5) | CA |
| PCR | | (SEQ ID NO: 6) |
| MAP2K1 | CCTTCCTCCCTCTTTCTTTCA | AGGCTGAGAGGGTGTCAC |
| exon 3 | (SEQ ID NO: 7) | AT |
| Sequenc- | | (SEQ ID NO: 8) |
| ing | | |

Similar to previous reports, a BRAF V600E mutation was detected in 3 of the initially screened 8 cases. An E102_I103del mutation in MAP2K1 was identified in one BRAF wild-type case using the Ion Comprehensive Cancer panel and confirmed by Sanger sequencing. Two additional MAP2K1 mutations (c. 159_173del, p.F53_Q28delinsL and c. G140A, p. R47Q) were identified by Sanger sequencing of MAP2K1 exons 2 and 3 that were not identified using the Ion AmpliSeq Comprehensive Cancer Panel.

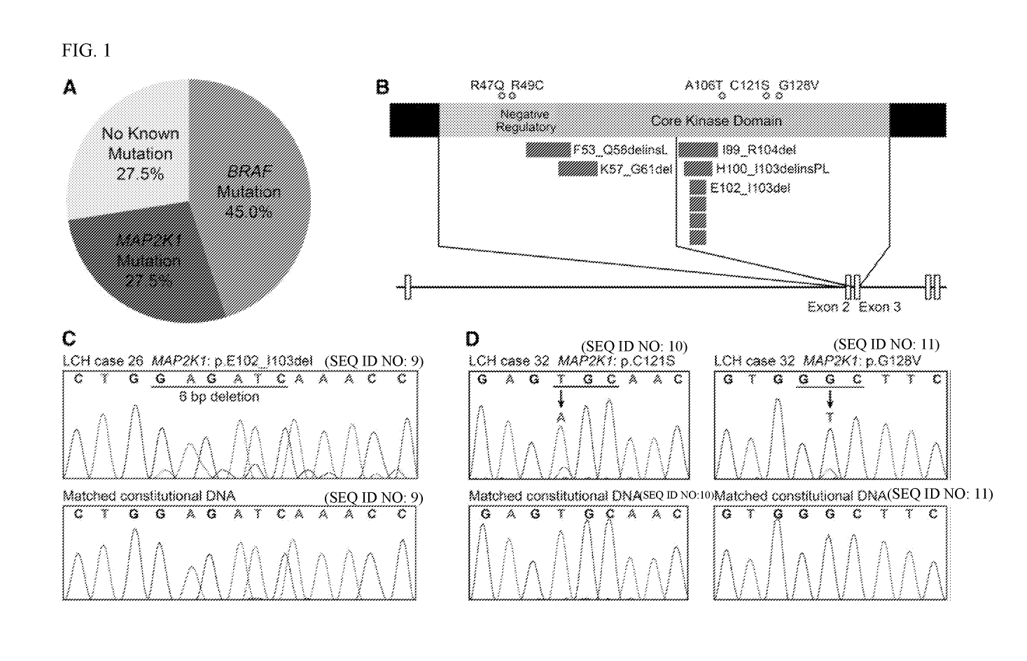

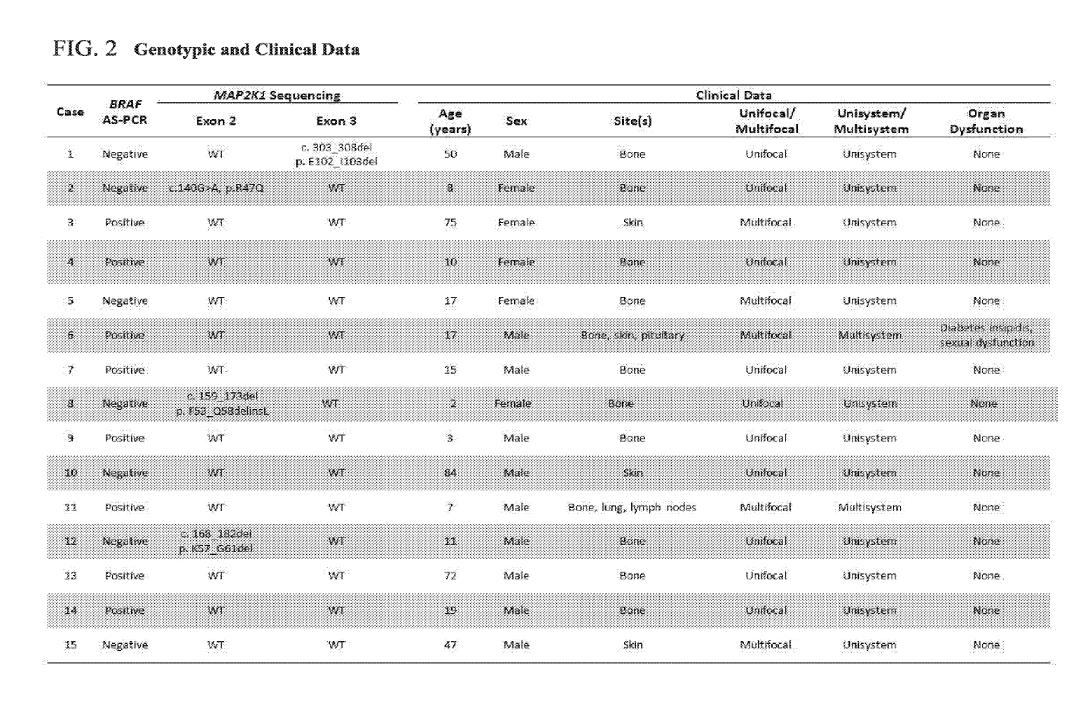

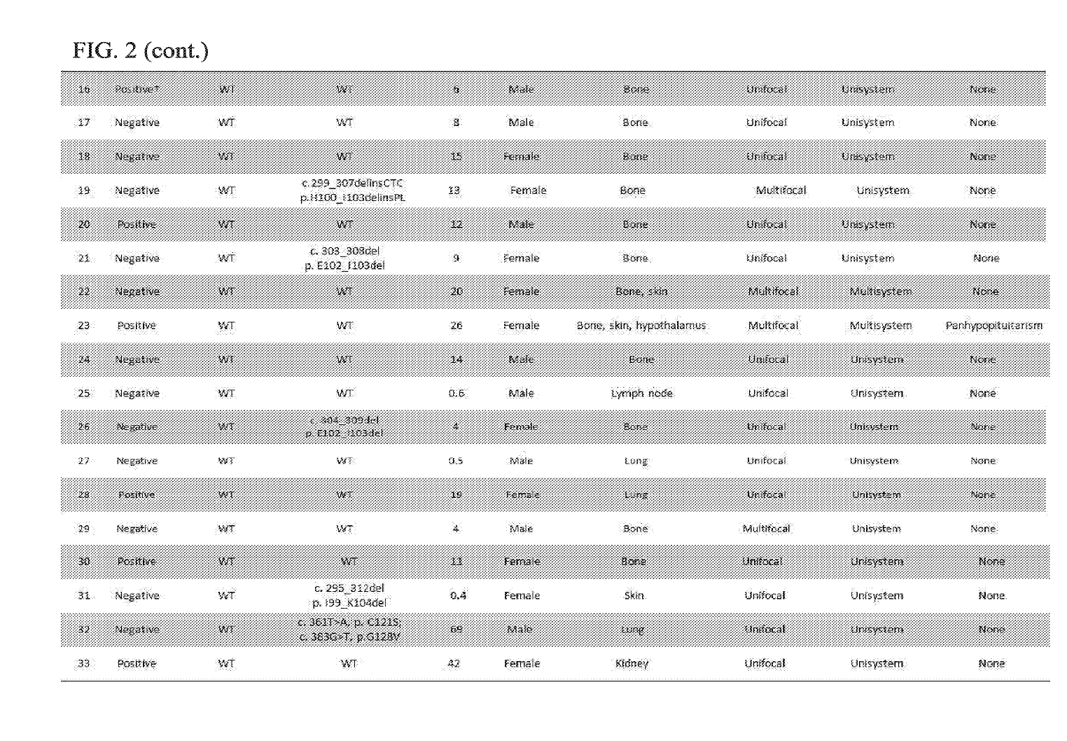

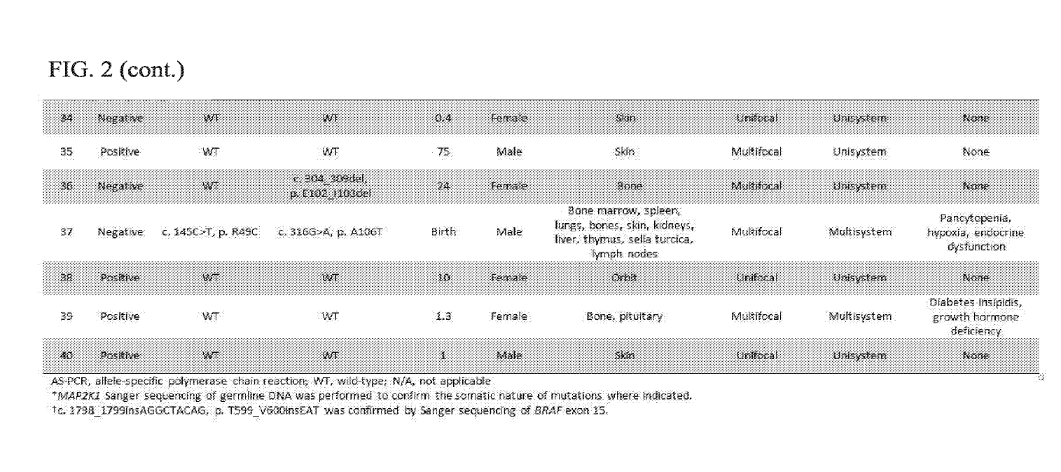

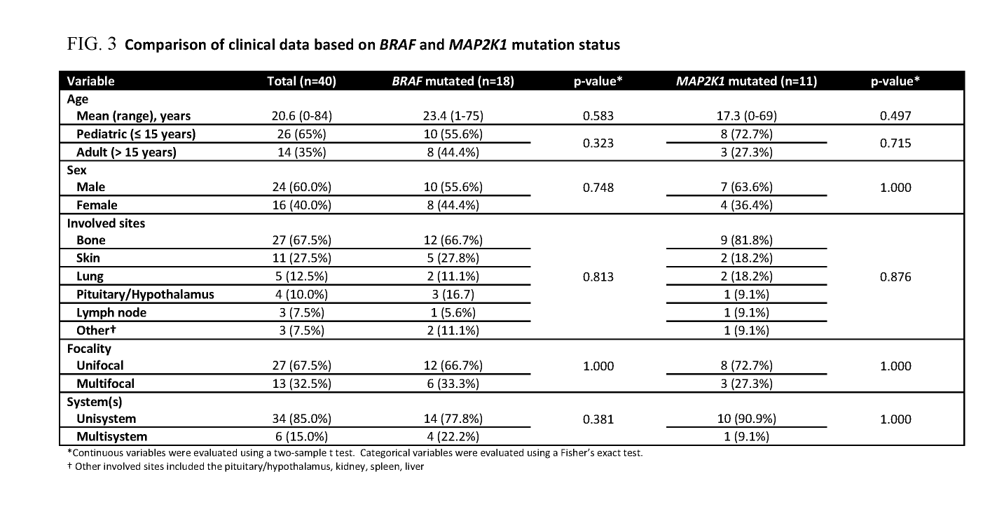

Analysis of 32 additional cases of LCH using BRAF V600E allele-specific PCR and Sanger sequencing of MAP2K1 exons 2 and 3 revealed BRAF and MAP2K1 mutations in a total of 18/40 (45.0%) and 11/40 (27.5%) cases, respectively (FIG. 1A). The MAPK2K1 mutations were mutually exclusive with BRAF mutations and were present in 11/22 (50%) BRAF wild-type cases. All MAP2K1 mutations were somatic based on sequencing of matched constitutional DNA. No statistically significant association was found between MAP2K1 mutation status and clinical indices such as age, sex, sites of involvement, or stage (FIGS. 2 and 3).

MAP2K1 encodes the dual specificity kinase MEK1 protein. MEK1 is normally activated by BRAF within the MAPK pathway and is directly upstream of extracellular signal-regulated kinases ERK1 and ERK2. MAP2K1 mutations have been described in several neoplasms including melanoma (see, e.g., Hodis E, et al., Cell. 2012; 150(2):251-63), lung carcinoma (see, e.g., Marks J L, et al., Cancer Res. 2008; 68(14):5524-8), and recently in BRAF V600E-negative hairy cell leukemia (see, e.g., Waterfall J J, et al., Nat Genet. 2014; 46(1):8-10). The identified MAP2K1 mutations were located in the negative regulatory region encoded by exon 2 and the catalytic core encoded by exon 3 (FIG. 1B; Table 2) (see, e.g., Bromberg-White J L, Brief Funct Genomics. 2012; 11(4):300-10; Fischmann T O, Biochemistry. 2009; 48(12):2661-74). Similar mutations affecting these sites have previously been demonstrated to result in constitutive activation of the MAPK pathway in non-hematologic neoplasms (see, e.g., Marks J L, et al., Cancer Res. 2008; 68(14):5524-8; Nikolaev S I, et al., Nat Genet. 2011; 44(2):133-9; Mansour S J, et al., Science. 1994; 265(5174):966-70; Wagle N, et al., J Clin Oncol. 2011; 29(22):3085-96). By comparison with the predominance of missense mutations observed in other neoplasms, the majority of MAP2K1 mutations in LCH were in-frame deletions. Six in-frame deletions involved exon 3 including residues E102 and 1103 (FIG. 1C). Two cases of mutations involving this site have been described in melanoma and lung adenocarcinoma (see, e.g., Hodis E, et al., Cell. 2012; 150(2):251-63; Marks J L, et al., Cancer Res. 2008; 68(14):5524-8). Another two deletions (F53_Q58delinsL and K57_G61del) occurred in exon 2 affecting the helix A regulatory region (see, e.g., Fischmann T O, et al., Biochemistry. 2009; 48(12):2661-74). Deletions involving this region have been reported to have 60-fold increased MEK1 enzymatic activity (see, e.g., Nikolaev S I, et al., Nat Genet. 2011; 44(2):133-9). Five missense mutations were also identified—R47Q, R49C, A106T, C121S and G128V. The C121S mutation has been shown to increase kinase activity and promote melanoma cell proliferation (see, e.g., Wagle N, et al., J Clin Oncol. 2011; 29(22):3085-96). Of note, two of the cases demonstrated two separate missense mutations at similar allele frequencies—one case with C121S and G128V (FIG. 1D) and one case with R49C and A106T. The former patient was diagnosed at birth, suffered aggressive multisystem disease refractory to several therapies, and died at 19 months of age (FIG. 2).

TABLE 2
BRAF V600E allele-specific PCR and MAP2K1
exons 2 and 3 Sanger sequencing results

| | | MAP2K1 Sanger Sequencing | | |
|---|---|---|---|---|
| | BRAF V600E | | | Confirmed |
| Case # | AS-PCR Result | Exon 2 | Exon 3 | Somatic* |
| 1 | Negative | WT | c. 303_308del | Yes |
| | | | p. E102_I103del | |
| 2 | Negative | c.140G > A, p.R47Q | WT | Yes |
| 3 | Positive | WT | WT | N/A |
| 4 | Positive | WT | WT | N/A |
| 5 | Negative | WT | WT | N/A |
| 6 | Positive | WT | WT | N/A |
| 7 | Positive | WT | WT | N/A |
| 8 | Negative | c. 159_173del | WT | Yes |
| | | p. F53_Q58delinsL | | |
| 9 | Positive | WT | WT | N/A |
| 10 | Negative | WT | WT | N/A |
| 11 | Positive | WT | WT | N/A |
| 12 | Negative | c. 168_182del | WT | Yes |
| | | p. K57_G61del | | |
| 13 | Positive | WT | WT | N/A |
| 14 | Positive | WT | WT | N/A |
| 15 | Negative | WT | WT | N/A |
| 16 | Positive† | WT | WT | N/A |
| 17 | Negative | WT | WT | N/A |
| 18 | Negative | WT | WT | N/A |
| 19 | Negative | WT | c.299_307delinsCTC | Yes |
| | | | p.H100_I103delinsPL | |
| 20 | Positive | WT | WT | N/A |
| 21 | Negative | WT | c. 303_308del | Yes |
| | | | p. E102_I103del | |
| 22 | Negative | WT | WT | N/A |
| 23 | Positive | WT | WT | N/A |
| 24 | Negative | WT | WT | N/A |
| 25 | Negative | WT | WT | N/A |
| 26 | Negative | WT | c. 304_309del | Yes |
| | | | p. E102_I103del | |
| 27 | Negative | WT | WT | N/A |
| 28 | Positive | WT | WT | N/A |
| 29 | Negative | WT | WT | N/A |
| 30 | Positive | WT | WT | N/A |
| 31 | Negative | WT | c. 295_312del | Yes |
| | | | p. I99_K104del | |
| 32 | Negative | WT | c. 361T > A, p. C121S; | Yes |
| | | | c. 383G > T, p.G128V | |
| 33 | Positive | WT | WT | N/A |
| 34 | Negative | WT | WT | N/A |
| 35 | Positive | WT | WT | N/A |
| 36 | Negative | WT | c. 304_309del, | Yes |
| | | | p. E102_I103del | |
| 37 | Negative | c. 145C > T, p. R49C | c. 316G > A, p. A106T | Yes |
| 38 | Positive | WT | WT | N/A |
| 39 | Positive | WT | WT | N/A |
| 40 | Positive | WT | WT | N/A |

AS-PCR, allele-specific polymerase chain reaction;
WT, wild-type;
N/A, not applicable
*MAP2K1 Sanger sequencing of germline DNA was performed to confirm the somatic nature of mutations where indicated.
†c. 1798_1799insAGGCTACAG, p. T599_V600insEAT was confirmed by Sanger sequencing of BRAF exon 15.

Because MEK1 is downstream of BRAF within the MAPK pathway, LCH patients with MAP2K1 mutations would not be expected to benefit from BRAF inhibitor therapy. Accordingly, MAP2K1 mutations have been demonstrated to confer resistance to BRAF inhibitor therapy in other neoplasms (see, e.g., Imielinski M, et al., Cell. 2012; 150(6):1107-20). Several small molecule inhibitors targeting MEK are FDA-approved or in clinical trials for the treatment of neoplasms with activating MAPK pathway mutations, principally BRAF mutated melanoma with and without MAP2K1 mutations (see, e.g., Emery CM, et al., Proc Natl Acad Sci USA. 2009; 106(48):20411-6; Flaherty K T, et al., N Engl J Med. 2012; 367(18):1694-703; Boers-Sonderen M J, et al., Anticancer Drugs. 2012; 23(7):761-4; Park S J, et al., Am J Med Sci. 2013; 346(6):494-8). However, at least one mutation identified in such experiments—C121S—has been shown to confer resistance to both BRAF inhibitors and current MEK inhibitors (see, e.g., Wagle N, et al., J Clin Oncol. 2011; 29(22):3085-96). Nevertheless, MEK1 and its downstream kinase ERK remain attractive targets for therapy in LCH.

In certain embodiments, the present invention provides methods for treating LCH comprising inhibiting MEK1 expression and/or activity in a subject (e.g., a human) having or at risk for having LCH. In some embodiments, inhibition of MEK1 expression and/or activity is accomplished through administration of a MEK1 inhibiting agent to the subject. Applicable examples of MEK1 inhibiting agents include, but are not limited to, Selumetinib (AZD6244) (see, e.g., Nature, 2012, 487(7408):505-9; Nature, 2010, 468(7326):973-7; Nature, 2010, 468(7326):968-72), PD0325901 (see, e.g., Nature, 2014, 10.1038/nature13887; Cell, 2012, 151(5):937-50; Nat Methods, 2011, 8(6):487-93), Trametinib (GSK1120212) (see, e.g., Nature, 2014, 510(7504):283-7; Nature, 2014, 10.1038/nature13887; Nature, 2014, 508(7494):118-22), U0126-EtOH (see, e.g., Cell, 2013, 153(4):840-54; Nat Genet, 2011, 44(2):133-9), PD184352 (C1-1040) (see, e.g., Science, 2011, 331(6019):912-6; Nat Genet, 2011, 44(2):133-9; Cancer Discov, 2013, 3(9):1058-71), Refametinib (RDEA119, Bay 86-9766), PD98059 (see, e.g., J Natl Cancer Inst, 2012, 104(21):1673-9; Hepatology, 2013, 59(4):1262-72; Sci Signal, 2011, 4(192):ra62), BIX 02189 (see, e.g., Anat Cell Biol, 2011, 44(4):265-73; Biochim Biophys Acta, 2014, 1843(5):945-54; Am J Pathol, 2013, 183(6):1758-68), Binimetinib (MEK162, ARRY-162, ARRY-438162) (see, e.g., Mol Oncol, 2014, 8(3):544-54), Pimasertib, SL-327, BIX 02188 (see, e.g., Mol Cancer, 2014, 13:40; Exp Neurol, 2012, 238(2):209-17), AZD8330 (see, e.g., Cell, 2012, 32(34):4034-42), TAK-733 (see, e.g., Mol Cancer Ther, 2014, 13(2)), Honokiol, and PD318088. In some In some embodiments, the treating further comprises administration of radiation therapy to the subject. In some embodiments, the treating further comprises administration of chemotherapy (e.g., alkylating agents, antimetabolites, vinca alkaloids, etc.) to the subject.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### Incorporation by Reference

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of assessing the Langerhans cell histiocytosis disease status of an individual, comprising: (a) evaluating a sample containing nucleic acids from the individual to detect the presence of one or more mutations in one or both alleles of the MAP2K1 gene, wherein evaluating comprises hybridizing to a MAP2K1 nucleic acid an oligonucleotide comprising a nucleotide sequence complementary with one or more MAP2K1 mutations, and (b) identifying the individual (i) as having Langerhans cell histiocytosis or being predisposed to Langerhans cell histiocytosis when the individual is homozygous for one or more MAP2K1 mutations, or (ii) as being predisposed to Langerhans cell histiocytosis when the individual is heterozygous for one or more MAP2K1 mutations,
wherein said sample is selected from the group consisting of blood, serum, and plasma.

2. The method of claim 1, wherein the one or more MAP2K1 mutations are nucleic acid sequence mutations in comparison to SEQ ID NO: 2 selected from the group consisting of 140G>A; 145C>T; 159_173del; 168_182del; 295_312del; 299_307delinsCTC; 303_308del; 316G>A; 304_309del; 361T>A; and 383G>T.

3. The method of claim 1, wherein said nucleic acid from the individual is RNA and the MAP2K1 nucleic acid is cDNA.

4. (canceled)

5. The method of claim 1, wherein said individual does not have a pathologic mutation in the BRAF gene, wherein said individual does not have a mutation in the BRAF gene encoding V600E mutation.

6-15. (canceled)

16. A method for detecting one or more MAP2K1 variants associated with Langerhans cell histiocytosis in a subject not having a BRAF gene encoding V600E mutation, comprising:
a) contacting a sample from a subject with a MAP2K1 variant detection assay under conditions that the presence of a MAP2K1 variant associated with Langerhans cell histiocytosis is determined; and
b) diagnosing said subject with Langerhans cell histiocytosis when one or more of said MAP2K1 variants are present in said sample,
wherein said one or more JAK/STAT pathway variants encodes a loss of function mutation, deletion mutation, insertion mutation, and/or a gain of function mutation,
wherein said subject is a human patient,
wherein said biological sample is selected from the group consisting of a tissue sample, a cell sample, and a blood sample.

17-18. (canceled)

19. The method of claim 16, wherein the MAP2K1 variant is a MAP2K1 nucleic acid variant selected from the group consisting of 140G>A; 145C>T; 159_173del; 168_182del; 295_312del; 299_307delinsCTC; 303_308del; 316G>A; 304_309del; 361T>A; and 383G>T.

20. The method of claim 16, wherein said determining comprises detecting variant MAP2K1 nucleic acids and/or MEK1 polypeptides.

21. The method of claim 20, wherein said detecting variant MAP2K1 nucleic acids comprises one or more nucleic acid detection method selected from the group consisting of sequencing, amplification and hybridization.

22. (canceled)

23. The method of claim 16, wherein said determining comprises a computer implemented method, wherein said computer implemented method comprises analyzing MAP2K1 variant information and displaying said information to a user.

24. (canceled)

25. The method of claim 16, further comprising the step of treating said subject for Langerhans cell histiocytosis under conditions such that at least one symptom of said Langerhans cell histiocytosis is diminished or eliminated, wherein said treating comprises inhibiting MEK1 expression and/or activity.

26. (canceled)

27. The method of claim 25, wherein said inhibiting MEK1 expression and/or activity is accomplished through administration of an agent configured to inhibit MEK1 expression and/or activity.

28. The method of claim 25, further comprising additionally administering radiation therapy and/or chemotherapy.

29. Use of a variant MAP2K1 nucleic acid or variant MEK1 polypeptide for detecting Langerhans cell histiocytosis in a subject.

30. The use of claim 29, wherein said MAP2K1 variant encodes a loss of function mutation, deletion mutation, insertion mutation, and/or a gain of function mutation.

31. The use of claim 29, wherein said subject is a human subject.

32. The use of claim 29,
wherein the MEK1 variant is a MEK1 amino acid variant selected from the group consisting of R47Q; R49C; F53_Q58delinsL; K57_G61del; 199_K104del; H100_I103delinsPL; E102_I103del; A106T; C121S; and G128V; and/or
wherein the MAP2K1 variant is a MAP2K1 nucleic acid variant selected from the group consisting of 140G>A; 145C>T; 159_173del; 168_182del; 295_312del; 299_307delinsCTC; 303_308del; 316G>A; 304_309del; 361T>A; and 383G>T.

33. The use of claim 29, wherein said determining comprises detecting variant MAP2K1 nucleic acids or variant MEK1 polypeptides.

34. The use of claim 33, wherein said detecting variant MAP2K1 nucleic acids comprises one or more nucleic acid detection method selected from the group consisting of sequencing, amplification and hybridization.

35-36. (canceled)
